(19) United States
(12) Patent Application Publication
(10) Pub. No.: US 2016/0183978 A1
(43) Pub. Date: Jun. 30, 2016

(54) CALLOUS, DEAD, DRY OR HARD SKIN REMOVER

(71) Applicant: Chibuzo Aniemena, Marina Del Rey, CA (US)

(72) Inventor: Chibuzo Aniemena, Marina Del Rey, CA (US)

(21) Appl. No.: 14/860,529

(22) Filed: Sep. 21, 2015

Related U.S. Application Data
(60) Provisional application No. 62/052,909, filed on Sep. 19, 2014.
(60) Provisional application No. 62/077,747, filed on Nov. 10, 2014.

(51) Int. Cl. A61B 17/54; A45D 29/00

(52) U.S. Cl. CPC A61B 17/54; A45D 29/00

(57) ABSTRACT

A callous, dead, dry or hard skin remover is disclosed. The remover comprises a hemispherical shaving head having a plurality of drilled holes and a sealing base, a collection compartment detachable to the sealing base of the hemispherical shaving head and is configured to collect skin residues removed by the hemispherical shaving head and a safety cap that covers the hemispherical shaving head when the remover is not used.

### CROSS-REFERENCE TO RELATED PATENT APPLICATIONS

Embodiments of the present invention relate to U.S. Provisional Application Ser. No. 62/052,909 filed Sep. 19, 2014, entitled “A Stainless Steel Compact Pedicure Foot File” and U.S. Provisional Application Ser. No. 62/077,747, filed Nov. 10, 2014. entitled “A General Purpose File/Grater/Shaver/Rasp Surface Perforated Holes Layout that can be manufactured for either food preparation or personal grooming”, the contents of which are incorporated by reference herein in their entirety and which are basis for a claim of priority.

### FIELD OF THE DISCLOSURE

The present disclosure generally relates to apparatuses for removing callous, dead, dry or hard skins. More specifically, the present disclosure relates to callous, dead, dry or hard skin removers that are semispherical in shape and provide multiple grating or shaving directions.

### BACKGROUND OF THE INVENTION

Conventional callous, dead. dry or hard skin removers (collectively as “callus remover(s)” hereafter) such as pedicure foot files, grater, shaver and/or rasp cannot shave off callous, dead, dry or hard skins from user effectively because they are either completely flat or slightly convex in shape. These shaped callus removers do not resist enough the natural cupping of the skin around the files when they are pressed against and are moved across the surface of the skin, which consequently reduce their effectiveness to shave off callous and dead skins.

In addition, conventional, callus removers only allow users to file, grate, shave and/or rasp skin in only one or two directions (i.e. a back and forth motion). The problem with these callus removers is the layout of the small elevations of the drilled holes which make up the surface of the callus removers are arranged in a way that only one row of elevations opposes the other row of elevations, which limit the directions that the user can file grate shave and/or rasp to only two (2) directions, which subsequently reduce optimum results of removing callous and dead skins.

Accordingly, there exists a need for improved callus remover that resists the natural cupping of the skin around the file when it is pressed against and is moved across the skin, resulting in a more superior shaving of callous and dead skin. There also exists a need for improved callus remover that provides users with multiple shaving directions, thereby achieving optimal shaving results.

### SUMMARY OF THE INVENTION

One objective of the present disclosure is to provide callous, dead, dry or hard skins removers that resist the natural cupping of the skin around the file when it is pressed against and is moved across the skin.

Another objective of the present disclosure is to provide callous, dead, dry or hard skins removers that provide users with multiple shaving directions.

In accordance with one aspect of at least one embodiment of the present disclosure, a callous, dead, dry or hard skin remover is disclosed comprising; a hemispherical shaving head having a plurality of drilled holes and a sealing base; a collection compartment detachable to the sealing base of the hemispherical shaving head and is configured to collect skin residues removed by the hemispherical shaving head; and a safety cap that covers the hemispherical shaving head when the remover is not used.

In another aspect of at least one embodiment of the present disclosure, each of the plurality of the drilled holes has an elevation on one of its side serving as the blade or cutter for removing callous, dead, dry or hard skins, In addition, the elevation for each of the plurality of drilled holes faces a direction that is different from the direction faced by the elevation of its adjacent drilling holes.

These and other features, aspects and advantages of the present invention will become better understood with reference to the following drawings, description and claims.

### BRIEF DESCRIPTION OF THE DRAWINGS

FIG. 1 is a schematic diagram according to an exemplary embodiment of the present invention.

FIG. 2 is a schematic diagram according to an exemplary embodiment of the present invention.

FIG. 3 is a schematic diagram according to an exemplary embodiment of the present invention.

FIG. 4 is a schematic diagram according to an exemplary embodiment of the present invention.

FIG. 5 is a schematic diagram according to an exemplary embodiment of the present invention.

FIG. 6A to 6D are schematic diagrams according to an exemplary embodiment of the present invention.

FIG. 7A and 7B are schematic diagrams according to an exemplary embodiment of the present invention.

FIG. 8 is a schematic diagram according to an exemplary embodiment of the present invention.

FIG. 9A and 9B are schematic diagrams according to an exemplary embodiment of the present invention.

FIG. 10 is a schematic diagram according to an exemplary embodiment of the present invention.

FIG. 11 is a schematic diagram according to an exemplary embodiment of the present invention.

FIG. 12 is a schematic diagram according to an exemplary embodiment of the present invention.

FIG. 13 is a schematic diagram according to an exemplary embodiment of the present invention.

FIG. 14 is a schematic diagram according to an exemplary embodiment of the present invention.

### DETAILED DESCRIPTION OF THE PREFERRED EMBODIMENT(S) AND INVENTION

The following description is not to be taken in a limiting sense, but is made merely for the purpose of illustrating the general principles of the disclosure, since the scope of the disclosure is best defined by the following claims. Various inventive features are described below that can each be used independently of one another or in combination with other features.

While the illustrations below primarily disclose apparatuses for removing callous, dead, dry or hard skins from a user, it should be noted that the apparatuses disclosed herein and their concepts can apply to any type of removers such as graters for foods (i.e., cheese graters) and are not limited to callus removers. As such, any removers using the same concept should also be considered as part of the present disclosure and shall entitle to the same scope of protection as the callus remover disclosed.

For purposes of the present disclosure, the term “remover” shall include apparatus and/or device, in any form and type, that is capable of provide filing, grating, shaving, exfoliating or rasping on anything such as skin and food. Examples of remover may include, but are not limited to, foot files, callus exfoliator, cheese grater, cuticle shaver/remover, pedicure rasp, vegetable shredders/slicers and everything else that are presently available or created in the future. The term “callus” should include callous, dead, dry and/or hard skins. The remover also includes manual handheld tools and their electrical versions. In addition, throughout the specification, terms like file, grate, shave, exfoliate and rasp are used interchangeably and shall have the same or similar meaning.

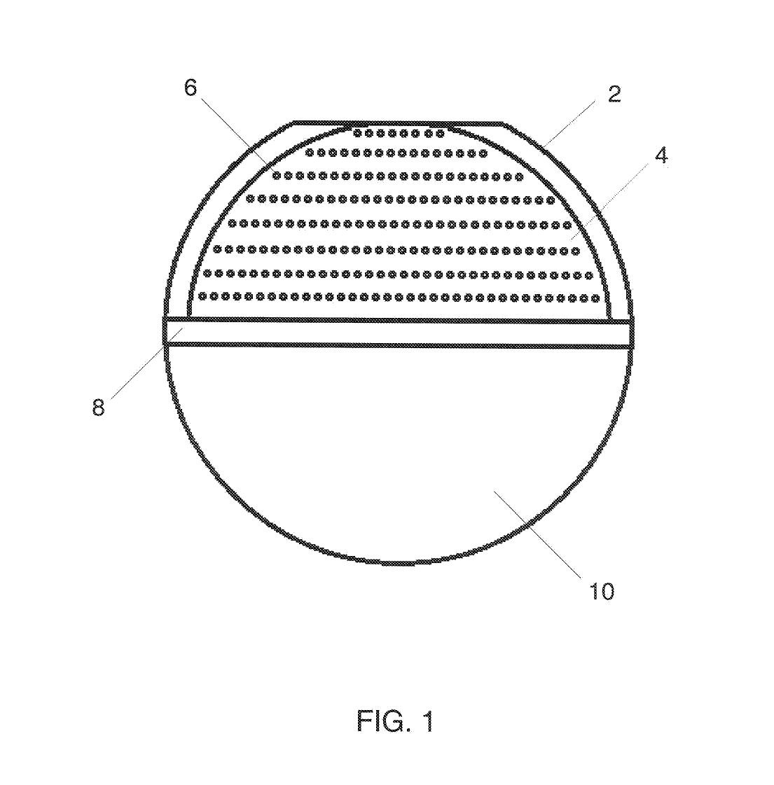

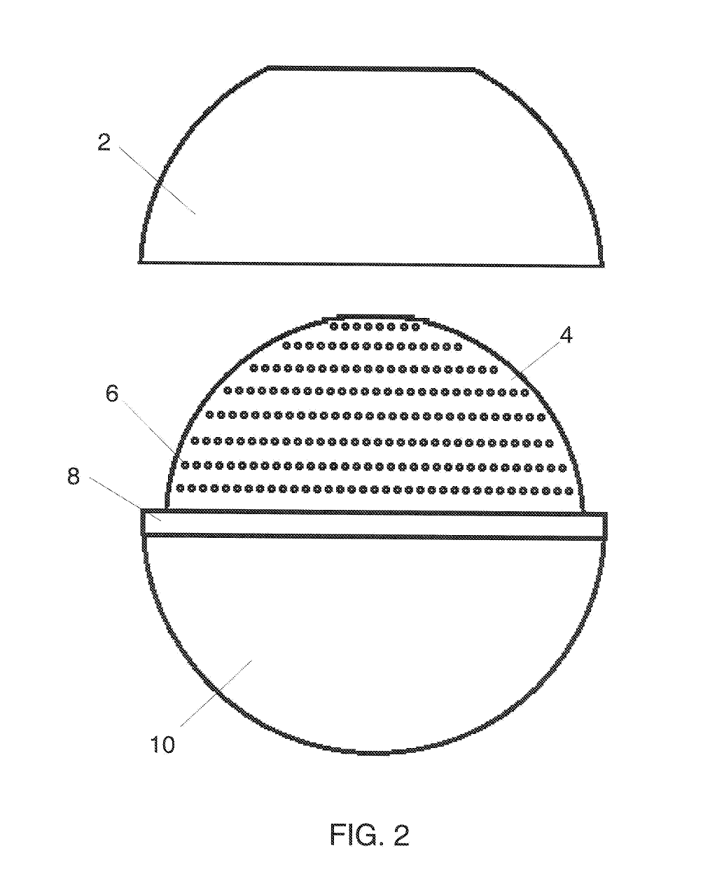

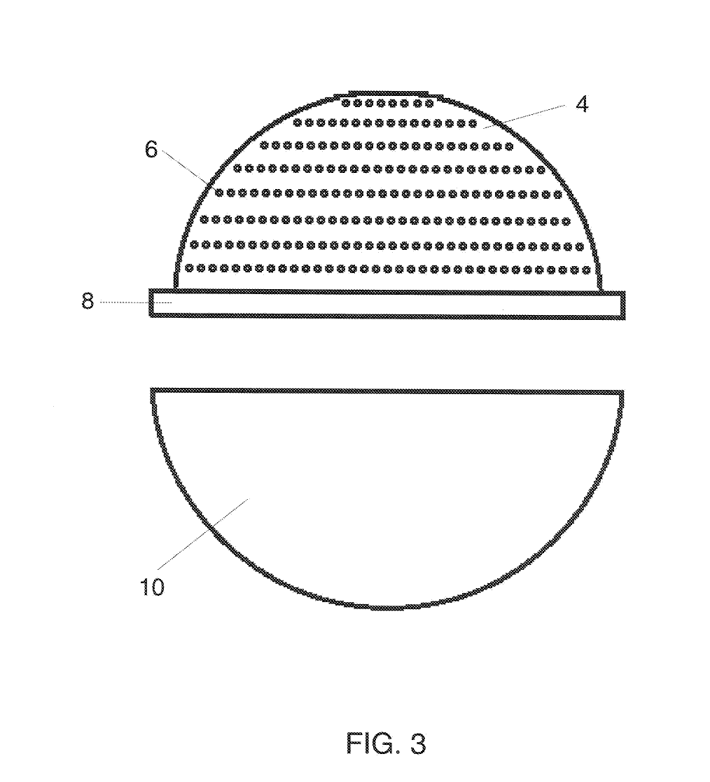

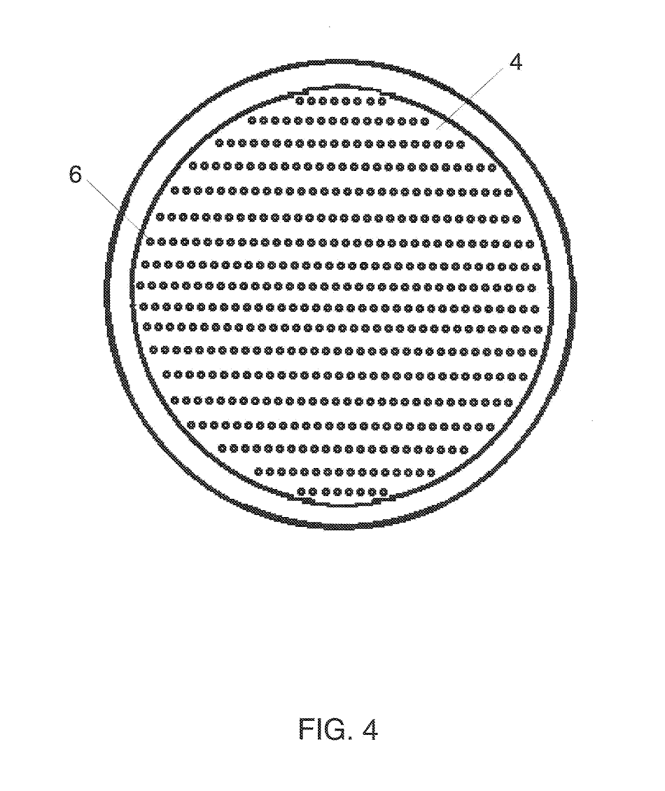

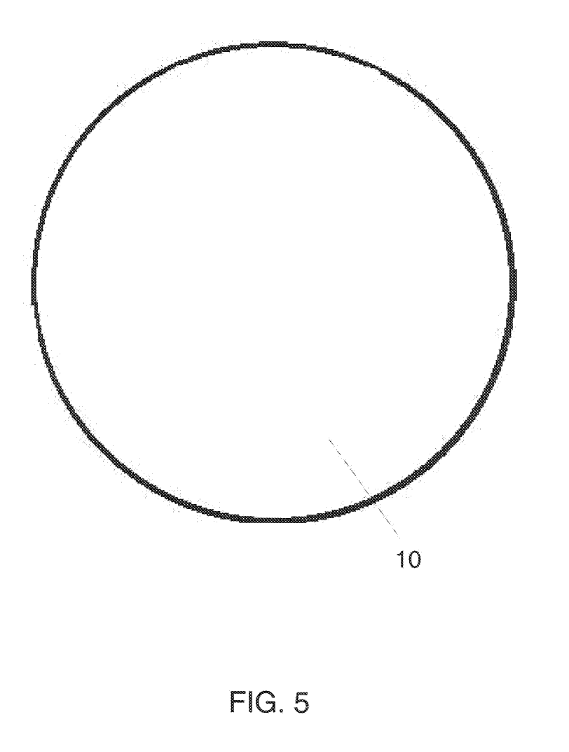

Broadly, embodiments of the present disclosure generally provide a callous, dead, dry or hard skin remover (collectively as “callus remover” hereafter). As shown by FIGS. 1 to 3, in one embodiment of the present disclosure, the disclosed callus remover comprises a hemispherical shaving head 4 having a plurality of drilled holes 6 and a sealing base 8; a hollow collection compartment 10 detachable to the sealing base 8 of the hemispherical shaving head 4 and is configured to collect skin residues removed by the hemispherical shaving head 4; and a safety cap 2 that covers the hemispherical shaving head 4 when the remover is not used (e.g., for safe storage). FIGS. 4 and 5 are the top view and the bottom view of the callus remover respectively.

The hemispherical shaving head can be made from any durable material such as metal, stainless steel and/or hard plastic. Preferably, the diameter of the hemispherical shaving head 4 is between 1 cm and 20 cm but it can be any size depending on which part of the body is the callus remover used.

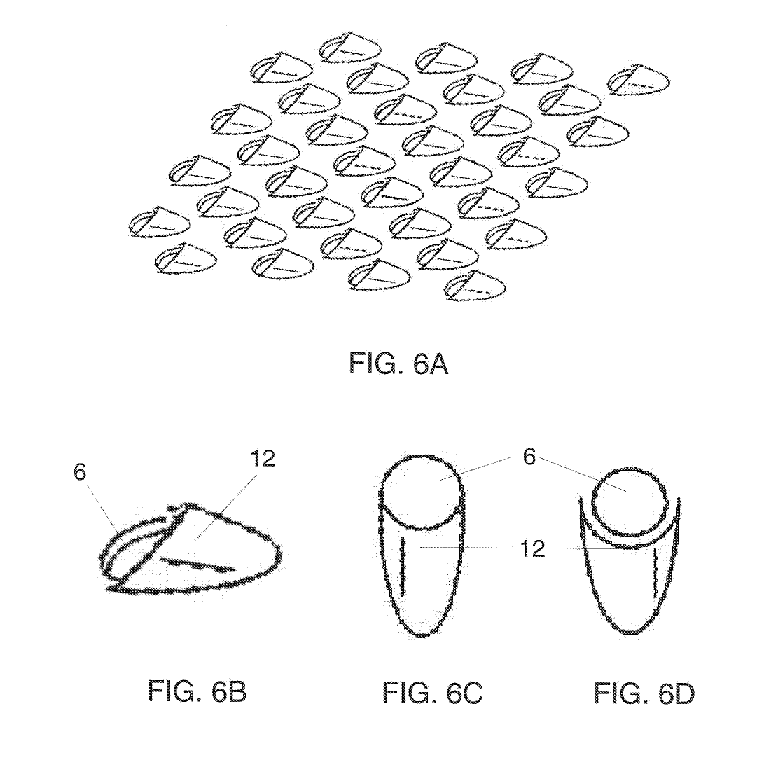

As shown by FIGS. 6A-6I), each of the plurality of drilled holes 6 on the hemispherical shaving head 4 has an elevation 12 on one of its side serving as the blade or cutter for removing callous, dead, dry or hard skins. As such, when a user press the callus remover of the present disclosure and move across the surface of his or her skin, the elevations 12 of the drilled holes 6 will remove calluses from the skin and the removed callus will fall through the drill holes 6 into the collection compartment 10. The taller or larger the elevation, the deeper or wider the elevation cuts on the surface of skin.

In yet another embodiment of the present disclosure, the collection compartment 10 is made from non-metallic material such as plastic or rubber, and is configured to attach to the sealing base 8 of the hemispherical shaving head 4 with clipping (e.g., squeeze and fit) or application torque (e.g., like cap and jar). Preferably, the attachment and detachment of collection compartment 10 and hemispherical shaving head 4 should be easy to allow the user to quickly empty and clean the removed calluses, from the collection compartment 10.

In yet another embodiment of the present disclosure, the collection compartment 10 is also hemispherical in shape. Thus, when the collection compartment 10 is attached to the hemispherical shaving head 10 with safety cap 2, they make almost a full sphere. The diameter of collection compartment 10 can be any size but preferably between 1 and 20 cm. so it can, also serve as a handle for the callus remover.

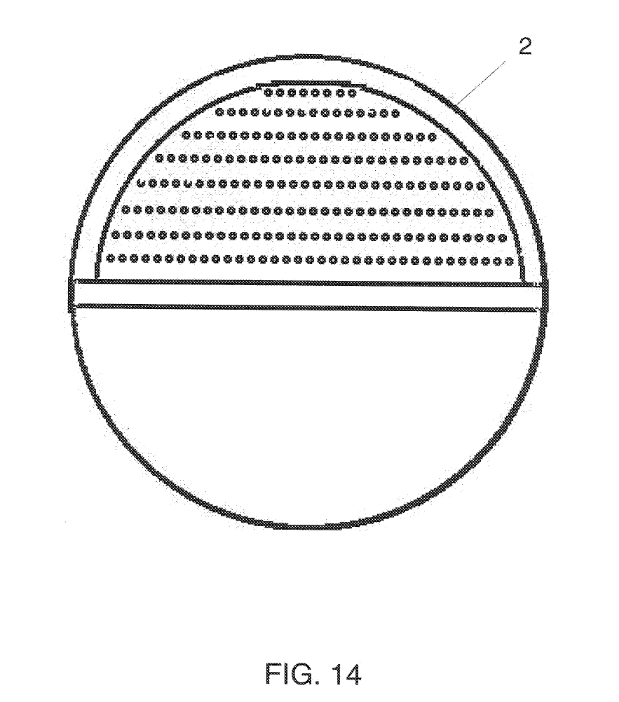

In yet another embodiment of the present disclosure, as shown by FIG. 14, both the collection compartment 10 and the safety cap 2 are hemispherical in shape. Thus, when the collection compartment 10 is attached to the hemispherical shaving head 10 with safety cap 2, they make a full sphere. The diameter of collection compartment 10 and the safety cap 2 can be any size but preferably between 1 and 20 cm, more preferably between 5 and 15 cm.

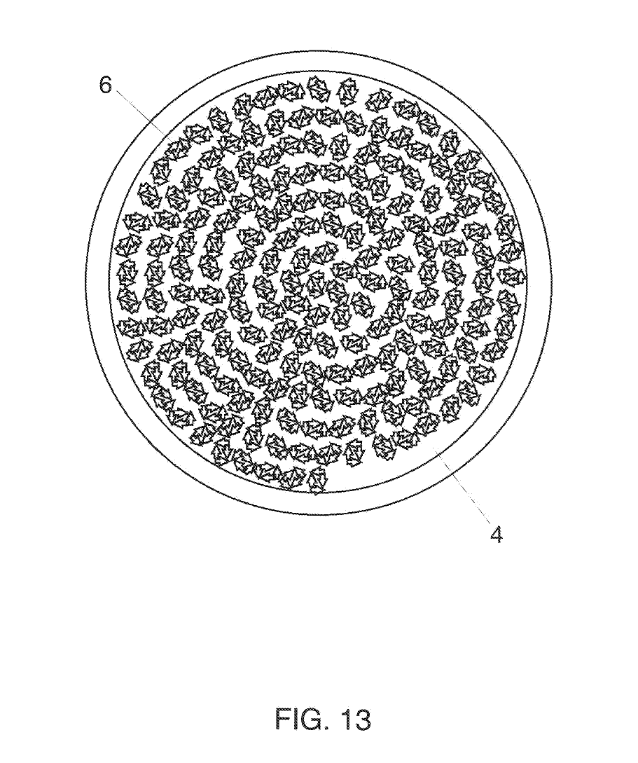

In yet another embodiment of the present disclosure, the plurality of drilled holes 6 are arranged in a spiral pattern from the center of the hemispherical shaving head center towards the sealing base (Refer to FIG. 13 for an example).

In yet another embodiment of the present disclosure, the plurality of drilled holes 6 are arranged in concentric circles.

In yet another embodiment of the present disclosure, the safety cap 2 has a flat surface allowing the callus remover of the present disclosure to stand when the safety cap 2 is capped on the metallic hemispherical shaving head 4. The safety cap 2 can optionally be translucent or having a color that matches the color of the collection compartment 10 to increase the callus remover's aesthetic appearance.

The hemispherical shaving head design allows the callus remover of the present disclosure to resists the natural cupping of the skin around the file/shaver when it is pressed against and moved across the user's skin, one could say “rollover the skin”, enabling the file/shaver to effectively shave or exfoliate callous and dead skin away at all areas of the user's body more effectively than conventional callus remover, making it ergonomic to the skin and resulting in better effectiveness.

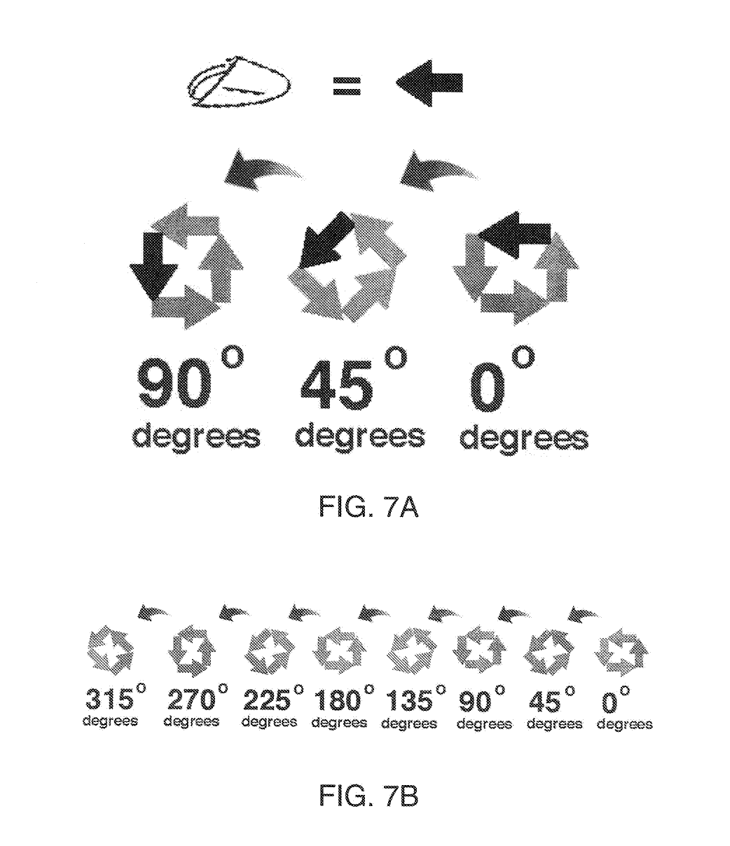

In yet another embodiment of the present disclosure, as shown by FIGS. 7-10, a callus remover that provides multiple directions of shaving is disclosed. The callus remover comprises a shaving head perforated with plurality of drilled holes where each of the plurality of drilled holes has an elevation on one of its side serving as the blade or cutter for removing callus. The elevation for each of the plurality of drilled holes faces a direction that is different from the direction faced by the elevation of its adjacent drilling holes. Preferably, the difference between the directions faced by the elevation of one drilled hole and the elevation of its adjacent drilled hole is between 1 and 90 degrees. The smaller the angle. the more shaving directions the callus remover can provide. In addition, the shaving, head can be flat, slightly convex or hemispherical in shape and can apply on all types of removers such as files, graters, shavers, exfoliator and rasps. Please note that the angles between elevations of drilling holes that are adjacent to each other do not have to be the same throughout the shaving head. They can be random or with a fixed pattern (e.g., 10 degrees between first and second elevations and 15 degrees between second and third elevations . . . etc). However, keeping angles uniform provides best result and is easier to implement and cheaper to manufacture. Similarly, the shaving head can be made from any durable material such as metal, stainless steel and/or hard plastic

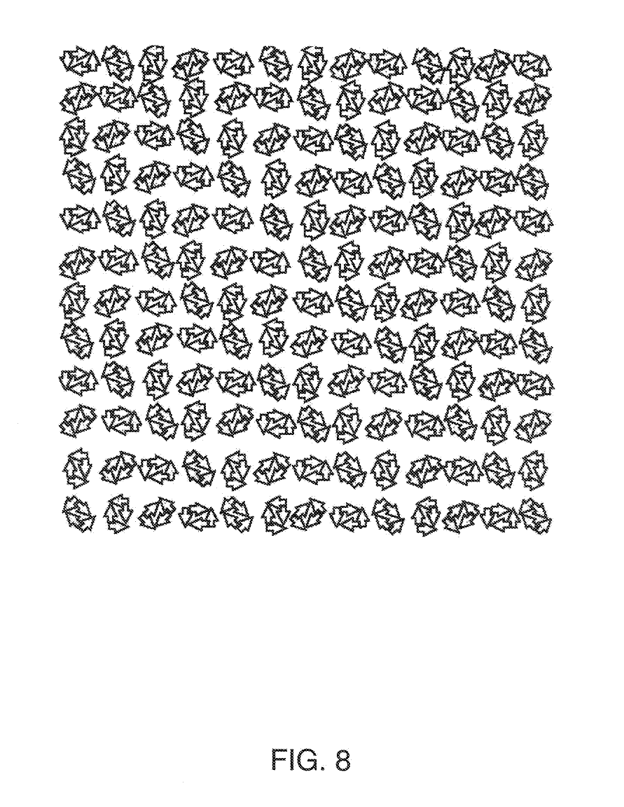

FIGS. 7A and 7B is an embodiment showing a callus remover or a shaving head that gives users eight (8) directions to file. grate, shave, exfoliate or rasp their skin (or vegetable if applied on kitchen appliance). Each arrow in the figure represents a drilled hole with an elevation and the direction of the arrow represents the direction faced by the elevation (i.e., the direction which the elevation cuts). In this embodiment, the elevation of each drilled hole is facing at a 45 degree angle from the elevation of the other drilled holes adjacent to it (i.e., to its left, right, above and below) and each drilled hole is laid in a group of 4 drilled holes with each group positioned at a 45 degrees angle to the group of 4 holes to its left and to its right. This formation is repeated throughout the shaving head as shown by FIG. 8. Note that the drilled holes does not have to be laid in a group of 4 holes, it can be 1 hole only or as many holes as the designer desire. As such, the configuration of drilling holes in this embodiment gives users eight (8) shaving directions (e.g., at 0, 45, 90, 135, 180, 225, 270 and 315 degrees), which is a great improvement over the conventional files, graters, shavers or raps which only enable their users two (2) directions to file, grate, shave, exfoliate and rasp food or skin (i.e., a back and forth motion at most) and thereby provide optimal shaving result.

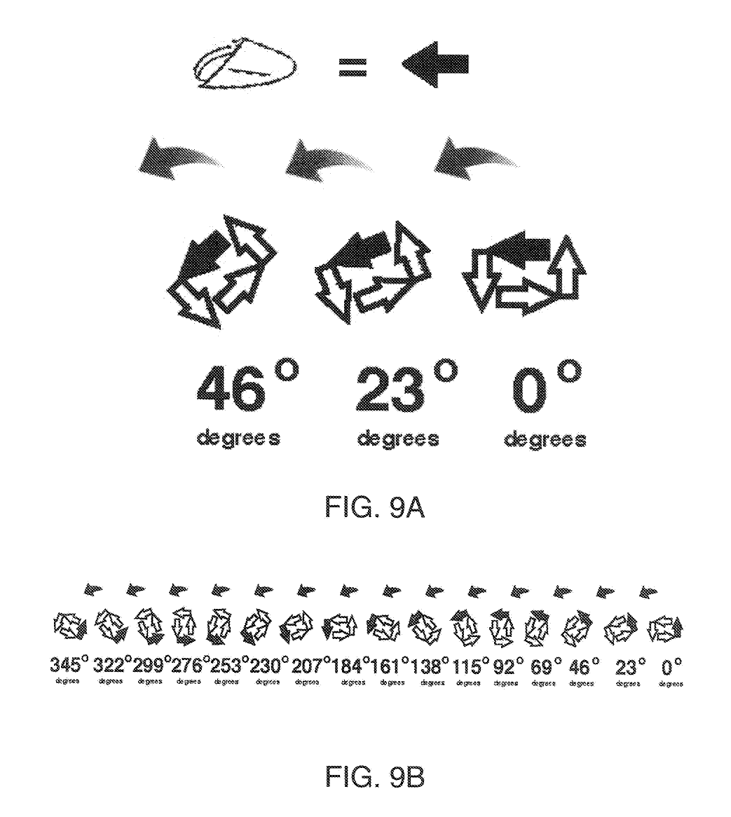

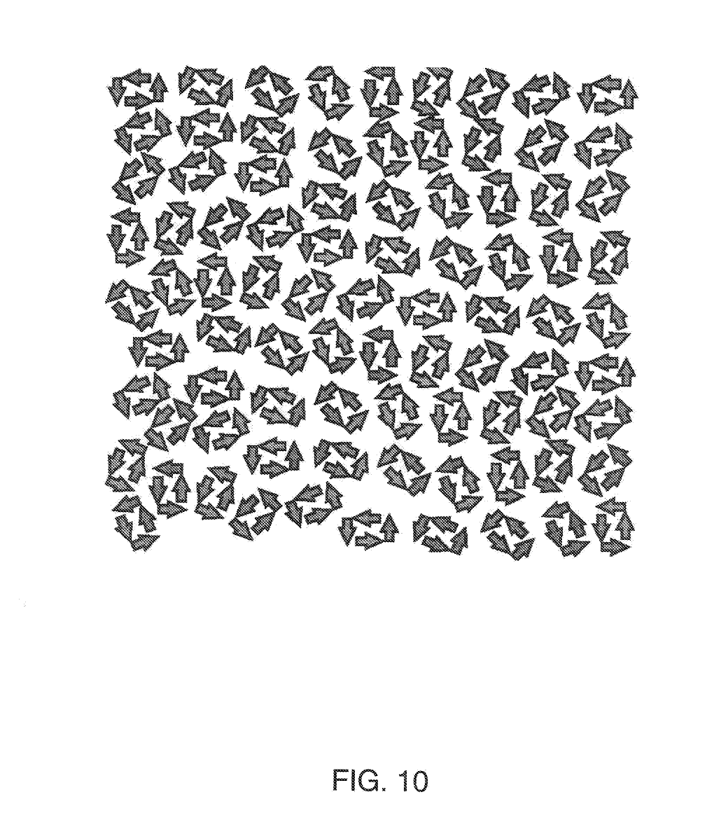

Similarly, FIGS. 9A and 9B illustrate another embodiment showing a callus remover or a shaving head that gives users sixteen (16) directions to file, grate, shave, exfoliate or rasp their skin (or vegetable if applied on kitchen appliance). In this embodiment, the elevation of each drilled hole is facing at a 23 degree angle from the elevation of the other drilled holes adjacent to it (i.e., to its left, right, above and below) and each drilled hole is again laid in a group of 4 drilled holes with each group positioned at a 23 degrees angle to the group of 4 holes to its left and to its right. This formation is repeated throughout the shaving head as shown by FIG. 10. As such, the configuration of drilling holes in this embodiment gives users sixteen (16) shaving directions (e.g., at 0, 23, 46, 69, 92, 115, 138, 161, 184, 207, 230, 253, 276, 299, 322 and 345 degrees).

Number of shaving directions with respect to the angle can simply be calculated by dividing 360 degree with the angle. For example, a 10 degree difference in angle between elevations will give user thirty-six (36) shaving directions (360/10). High number of shaving directions allows the user to shave their skin in all types of motions, i.e., circular or zigzag motion, while not compromising consistency or shaving results.

In yet another embodiment of the present disclosure, the shaving head with multiple shaving directions is also hemispherical, which allows the callus remover to additionally resists the natural cupping of the skin around the file/shaver when it is pressed against and moved across the user's skin. Thus the combination of hemispherical shaving head and multiple shaving directions enables the disclosed file/shaver to effectively shave callous and dead skin away at all areas of the user's body more effectively than conventional callus remover, making it ergonomic to the skin and resulting in better effectiveness.

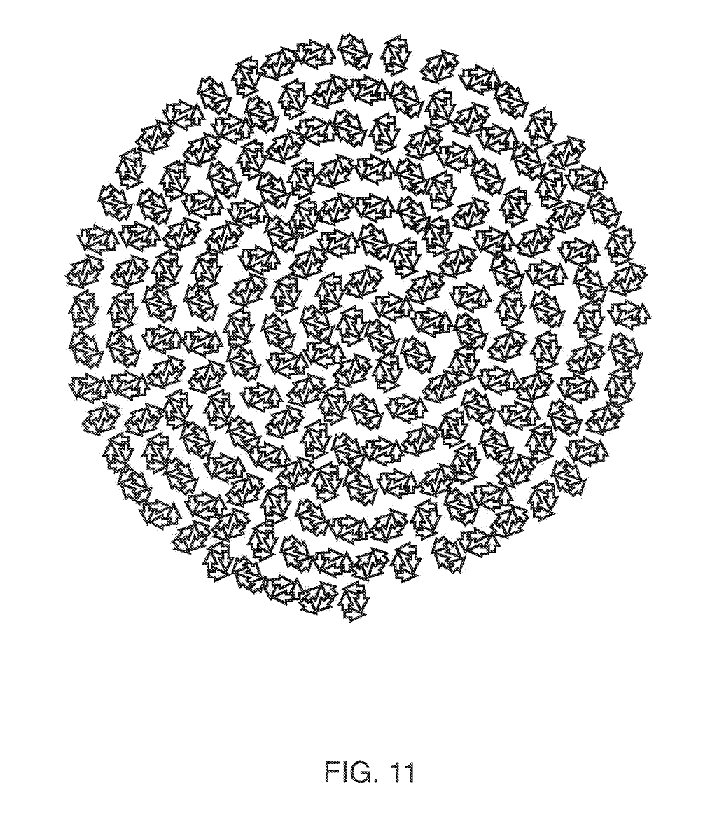

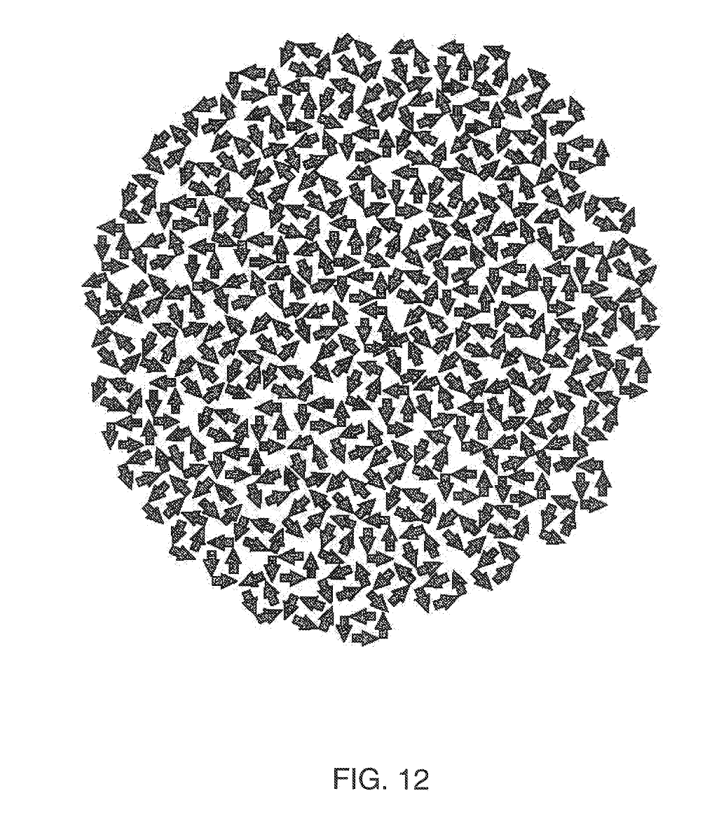

FIGS. 11 and 13 show an embodiment of a hemispherical shaving head with eight (8) shaving directions (45 degree) where as FIG. 12 shows another embodiment of hemispherical shaving head with sixteen (16) shaving directions (23 degree).

In general, shaving head that gives less direction (e.g., 8 directions) should have drilled holes that are bigger in size compared to the drilled holes of shaving head that gives more directions (e.g., 16 directions). The wider angle and larger drilled holes thus provide a wider elevation and larger shaving deposits while the narrower angle and smaller elevations of the drilled holes provide its user with finer shaving deposits.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples. but by all embodiments and methods within the scope and spirit of the invention as claimed.

What is claimed is:

1. A callous, dead, dry or hard skin remover, the remover comprising:
a hemispherical shaving head having a plurality of drilled holes and a sealing base;
a collection compartment detachable to the sealing base of the hemispherical shaving head and is configured to collect skin residues removed by the hemispherical shaving head; and
a safety cap that covers the hemispherical shaving head when the remover is not used.

2. The remover of claim 1, wherein the hemispherical shaving head is made of metal, hard plastic or stainless steel.

3. The remover of claim 1, wherein the hemispherical shaving head has a diameter between 1 cm and 20 cm.

4. The remover of claim 1, wherein the collection compartment is hemispherical and has a diameter between 1 cm and 20 cm.

5. The remover of claim 1, wherein each of the plurality of drilled holes has an elevation on one of its side serving as a blade or cutter for removing callous, dead, dry or hard skins.

6. The remover of claim 5, wherein the elevation for each of the plurality of drilled holes faces a direction that is different from the direction faced by the elevation of its adjacent drilling holes.

7. The remover of claim 6, wherein the difference in directions faced by each of the plurality of drilled holes and its adjacent drilled hole is between 1 and 90 degrees.

8. The remover of claim 1, where the plurality of drilled holes are arranged in a spiral pattern from hemispherical shaving head's center towards the sealing base.

9. The remover of claim 1, wherein the collection compartment attached to the sealing base of the hemispherical shaving head through clipping or application torque.

10. A callus remover, the remover comprising:
a hemispherical shaving head perforated with plurality of drilled holes that are drilled in a spiral pattern from the hemispherical shaving head's center going out towards its base;
a collection compartment detachable to the hemispherical shaving head which collects callus removed by the hemispherical shaving head; and
a removable safety cap that covers the hemispherical shaving head when the remover is not used.

11. The remover of claim 10, wherein the hemispherical shaving head is made of metal, hard plastic or stainless steel.

12. The remover of claim 10, wherein the hemispherical shaving head has a diameter between 1 cm and 20 cm.

13. The remover of claim 10, wherein the collection compartment is hemispherical and has a diameter between 1 cm and 20 cm.

14. The remover of claim 10, wherein each of the plurality of drilled holes has an elevation on one of its side serving as the blade or cutter for removing callous, dead, dry or hard skins.

15. The remover of claim 14, wherein the elevation for each of the plurality of drilled holes faces a direction that is different from the direction faced by the elevation of its adjacent drilling holes.

16. The remover of claim 14, wherein the difference in directions faced by each of the plurality of drilled holes and its adjacent drilled hole is between 1 and 90 degrees.

17. A callus remover, the remover comprising:
a metallic hemispherical shaving head perforated with plurality of drilled holes that are drilled in a spiral pattern from the hemispherical shaving head's center going out towards its base, wherein each of the plurality of drilled holes has an elevation on one of its side serving as the blade or cutter for removing callus and the elevation for each of the plurality of drilled holes faces a direction that is different from the direction faced by the elevation of its adjacent drilling holes;
a hemispherical and hollow collection compartment having a diameter between 1 to 20 cm detachable to the hemispherical shaving head and is configured in a way that it collects and stores callus removed by the hemispherical shaving head; and
a safety cap that covers the hemispherical shaving head when the remover is not used.

18. The remover of claim 1, wherein the hemispherical and hollow collection compartment attached to the metallic hemispherical shaving head through clipping or application torque.

19. The remover of claim 1, wherein each drilled hole is laid in a group of 4 drilled holes with each group positioned at a fix degrees angle to the group of 4 holes to its adjacent

[symbol]

20. The remover of claim 1, wherein the safety cap has a flat surface allowing the remover to stand when the safety cap is capped on the metallic hemispherical shaving head.
